(19) United States
(12) Patent Application Publication
(10) Pub. No.: US 2009/0306081 A1
(43) Pub. Date: Dec. 10, 2009

(54) Solid Dosage Formulations of Hydralazine Compounds and Nitric Oxide Donor Compounds

(76) Inventor: L. Gordon Letts, Dover, MA (US)

Correspondence Address: WILMERHALE/NITROMED, 1875 PENNSYLVANIA AVE, NW, WASHINGTON, DC, 20006, US

(21) Appl. No.: 12/300,593

(22) PCT Filed: May 16, 2007
(86) PCT No.: PCT/US07/11655

§ 371 Date: May 6, 2009

Related U.S. Application Data
(60) Provisional application No. 60/800,718, filed on May 16, 2006.
(60) Provisional application No. 60/841,569, filed on Sep. 1, 2006.

(51) Int. Cl. A61K 31/50; A61P 9/08

(52) U.S. Cl. 514/247

(57) ABSTRACT

The invention provides solid dosage formulations, methods of making and using the formulations comprising at least one hydralazine compound or a pharmaceutically acceptable salt thereof, and at least one excipient or carrier, wherein the formulations have less than about 0.001% to about 0.1% of a hydrazone compound based on the total weight of the formulation. The invention also provides solid dosage formulations, methods of making and using the formulations comprising at least one hydralazine compound or a pharmaceutically acceptable salt thereof, and at least one nitric oxide donor compound or a pharmaceutically acceptable salt thereof and at least one excipient or carrier, wherein the formulations have less than about 0.001% to about 0.1% of a hydrazone compound based on the total weight of the formulation.

### RELATED APPLICATIONS

This application claims priority under 35 USC § 119 to U.S. Application No. 60/800,718 filed May 16, 2006 and U.S. Application No. 60/841,569 filed Sep. 1, 2006; the disclosure of each of which is incorporated by reference herein in its entirety.

### FIELD OF THE INVENTION

The invention provides solid dosage formulations, methods of making and using the formulations comprising at least one hydralazine compound or a pharmaceutically acceptable salt thereof, and at least one excipient or carrier, wherein the formulations have less than about 0.0011% to about 0.1% of a hydrazone compound based on the total weight of the formulation. The invention also provides solid dosage formulations, methods of making and using the formulations comprising at least one hydralazine compound or a pharmaceutically acceptable salt thereof, and at least one nitric oxide donor compound or a pharmaceutically acceptable salt thereof and at least one excipient or carrier, wherein the formulations have less than about 0.001% to about 0.1% of a hydrazone compound based on the total weight of the formulation. The excipients or carriers of the formulation are non-reducing sugars such as, for example, cellulose, microcrystalline cellulose, mannitol and sorbitol. The solid dosage hydralazine formulations further comprise at least one chelating agent, at least one acidic agent and have a water content of about 1% to about 3% based on the total weight of the formulation.

### BACKGROUND OF THE INVENTION

Hydralazine hydrochloride is a peripheral vasodilator discovered about 50 years ago that exerts an antihypertensive effect directly on vascular smooth muscle producing relaxation of muscle fibers resulting in a decrease in blood pressure. Hydralazine is extensively metabolized in the body to products that are excreted predominantly in the urine, and undergoes N-acetylation, oxidation, hydroxylation, hydrazone formation and conjugation.

Hydralazine hydrochloride is very unstable in all of the injectable pharmaceutical formulations currently commercially available. Continuing instability problems with injectable hydralazine hydrochloride, for example, have plagued pharmaceutical manufacturers for many years, forcing these companies to remove their injectable hydralazine products from the marketplace. Although a shelf life of 12 months is currently required for FDA approval for injectable hydralazine hydrochloride, only a few companies have been able to satisfy this requirement with adequate stability data.

Hydralazine hydrochloride also undergoes several pharmaceutically undesirable reactions such as chelation with metal ions, oxidation, and pH-dependent decomposition. It is believed that these reactions, which often cause discoloration of hydralazine compositions, are also due to the highly reactive hydrazino group.

Despite its unique pharmacologic properties as a hypertensive drug, the therapeutic use of hydralazine hydrochloride has been limited by its instability during storage and difficulties in handling. A stable solid dosage hydralazine formulation that is easily manufactured and does not degrade during extended storage represents a significant advance.

There is a need in the art for stable solid dosage formulations comprising hydralazine compounds. The invention is directed to these, as well as other, important ends.

### SUMMARY OF THE INVENTION

The invention provides solid dosage formulations comprising at least one hydralazine compound or a pharmaceutically acceptable salt thereof and at least one excipient or carrier, wherein the formulations have less than about 0.001% to about 0.1% of a hydrazone compound based on the total weight of the formulation. The solid dosage formulation further comprises at least one chelating agent and at least one acidic agent. The solid dosage formulation is essentially free of metal ions and has a water content of about 1% to about 3% based on the total weight of the formulation. In one embodiment the hydralazine compound or pharmaceutically acceptable salt thereof is hydralazine hydrochloride. In another embodiment the excipients or carriers are non-reducing sugars, such as, for example, cellulose, microcrystalline cellulose, mannitol and sorbitol.

The invention provides solid dosage formulations comprising at least one hydralazine compound or a pharmaceutically acceptable salt thereof, and at least one nitric oxide donor compound or a pharmaceutically acceptable salt thereof, and at least one excipient or carrier, wherein the formulations have less than about 0.001% to about 0.1% of a hydrazone compound based on the total weight of the formulation. The solid dosage formulation further comprises at least one chelating agent and at least one acidic agent. The solid dosage formulation is essentially free of metal ions and has a water content of about 1% to about 3% based on the total weight of the formulation. In one embodiment the hydralazine compound or pharmaceutically acceptable salt thereof is hydralazine hydrochloride. In another embodiment the nitric oxide donor is isosorbide dinitrate (ISDN) and isosorbide mononitrate (ISMN). In another embodiment the nitric oxide donor is isosorbide dinitrate (ISDN). In another embodiment, the excipients or carriers are non-reducing sugars such as, for example, cellulose, microcrystalline cellulose, mannitol and sorbitol.

The invention provides methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; (s) treating respiratory disorders; (t) treating blood disorders; (u) treating the symptoms and/or complications associated with blood disorders; (v) treating preeclampsia; by administering to the patient in need thereof an effective amount of the formulations.

The invention is described in more detail below.

### DETAILED DESCRIPTION OF THE INVENTION

As used throughout the disclosure, the following terms, unless otherwise indicated, shall be understood to have the following meanings.

“Patient” refers to animals, preferably mammals, most preferably humans, and includes males and females.

“Heart failure” includes, but is not limited to congestive heart failure, compensated heart failure, decompensated heart failure, and the like.

“Compensated heart failure” refers to a condition in which the heart functions at an altered, but stable physiologic state, e.g. at a different but stable point on the Frank-Starling-curve through an increase in preload or after development of myocardial hypertrophy. Compensated heart failure can result in multiple complications, such as progressive increase in capillary related edema, progressive renal failure, or progressive ischemic tissue damage.

“Decompensated heart failure” refers to a condition in which the heart functions at an altered and unstable physiologic state in which cardiac function and related or dependent physiologic functions deteriorate progressively, slowly or rapidly. Decompensated heart failure can result in multiple complications, such as progressive increase in capillary related edema, progressive renal failure, or progressive ischemic tissue damage.

“Reducing hospitalizations related to heart failure” includes but is not limited to prolonging time to hospitalization for heart failure; prolonging time to first hospitalization for heart failure; reducing the total number of days a patient with heart failure spends in the hospital for heart failure for a single hospital stay (i.e., reducing the duration of a single hospital stay for a patient with heart failure); reducing the total number of days a patient spends in the hospital for heart failure for multiple hospital stays (i.e., two or more hospital stays); reducing the number of hospital admissions for heart failure; and the like.

“Oxygen consumption” can be measured during a progressive maximal bicycle-ergometer exercise test taken while the expired air is collected continuously to monitor oxygen consumption. Dyspnea or fatigue typically occurs at a peak oxygen consumption of <25 ml per kilogram of body weight per minute. Patients with pulmonary diseases, obstructive valvular diseases and the like, tend to have a low oxygen consumption. An increase in a patient's oxygen consumption typically results in the patient's increased exercise tolerance and would imply that the patient would have an improved quality of life.

“Quality of life” refers to one or more of a person's ability to walk, climb stairs, do errands, work around the house, participate in recreational activities, and/or not requiring rest during the day, and/or the absence of sleeping problems or shortness of breath. The quality of life can be measured using the Minnesota Living with Heart Failure questionnaire. The questionnaire is self-administered after brief standardization instructions. The score is obtained by summing the ranks of the responses to each question.

“Cardiovascular disease or disorder” refers to any cardiovascular disease or disorder known in the art, including, but not limited to, heart failure, restenosis, hypertension (e.g. pulmonary hypertension, systolic hypertension, labile hypertension, idiopathic hypertension, low-renin hypertension, salt-sensitive hypertension, low-renin, salt-sensitive hypertension, thromboembolic pulmonary hypertension; pregnancy-induced hypertension; renovascular hypertension; hypertension-dependent end-stage renal disease, hypertension associated with cardiovascular surgical procedures, hypertension with left ventricular hypertrophy, and the like), diastolic dysfunction, coronary artery disease, myocardial infarctions, cerebral infarctions, arterial stiffness, atherosclerosis, atherogenesis, cerebrovascular disease, angina, (including chronic, stable, unstable and variant (Prinzmetal) angina pectoris), aneurysm, ischemic heart disease, cerebral ischemia, myocardial ischemia, thrombosis, platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiograph, vascular grafting, coronary artery bypass surgery, thromboembolic events, post-angioplasty restenosis, coronary plaque inflammation, hypercholesterolemia, embolism, stroke, shock, arrhythmia, atrial fibrillation or atrial flutter, thrombotic occlusion and reclusion cerebrovascular incidents, left ventricular dysfunction and hypertrophy, and the like.

“Respiratory disorder” refers to any respiratory disease or respiratory disorder, such as, for example, chronic obstructive pulmonary disease, pulmonary hypertension, emphysema, asthma, cystic fibrosis and bronchitis, acute pulmonary vasoconstriction, pneumonia, traumatic injury, aspiration or inhalation injury, fat embolism in the lung, acidosis, inflammation of the lung, adult respiratory distress syndrome, acute pulmonary edema, acute mountain sickness, post cardiac surgery, pulmonary hypertension, persistent pulmonary hypertension of the newborn, perinatal aspiration syndrome, hyaline membrane disease, acute pulmonary thromboembolism, heparin-protamine reactions, sepsis, status asthmaticus, hypoxia, pulmonary hypertension, bronchopulmonary dysplasia, chronic pulmonary thromboembolism, idiopathic pulmonary hypertension, primary pulmonary hypertension, chronic hypoxia, sarcoidosis, idiopathic pulmonary fibrosis, pneumonitis, postperfusion lung, dyspnea, acute and chronic cough, pneumothorax, alveolar hyperventilation disorders, interstitial lung disease, pneumoconiosis, pneumocystosis, lung tumors, inflammatory respiratory disease, including, but not limited to (acute) respiratory disease syndrome (ARDS), IRDS, severe acute respiratory disease (SARS), porcine reproductive and respiratory syndrome (PRRS), porcine epidemic abortion and respiratory syndrome (PEARS), swine infertility and respiratory syndrome (SIRS) and the like.

“Blood disorder” refers to any disorder related to blood, including, but not limited to, sickle cell anemia, thalassemia, hemoglobin C disease, hemoglobin H disease, hemoglobin SC disease, sickle thalassemia, hereditary spherocytosis, hereditary elliptocytosis, hereditary ovalcytosis, glucose-6-phosphate deficiency and other red blood cell enzyme deficiencies, paroxysmal nocturnal hemoglobinuria (PNH), paroxysmal cold hemoglobinuria (PCH), thrombotic thrombocytopenic purpura/hemolytic uremic syndrome (TTP/HUS), idiopathic autoimmune hemolytic anemia, drug-induced immune hemolytic anemia, secondary immune hemolytic anemia, non-immune hemolytic anemia caused by chemical or physical agents, malaria, falciparum malaria, bartonellosis, babesiosis, clostridial infection, severe haemophilus influenzae type B infection, transfusion reaction, ryabdomyolysis (myoglobinemia), transfusion of aged blood, cardiopulmonary bypass, hemodialysis, and the like.

“Symptoms and/or complications resulting from a blood disorder” includes, but is not limited to, decreased blood flow, peripheral vascular disease, pulmonary hypertension, including, but not limited to, neonatal pulmonary hypertension, primary pulmonary hypertension, secondary pulmonary hypertension, and the like; cutaneous ulceration, acute renal failure, chronic renal failure, intravascular thrombosis, systemic systolic hypertension, oxidative stress, endothelial dysfunctions, jaundice, hemorrhaging, organ dysfunction, fatigue, shortness of breath, tissue damage due to hypoxia, ischemia, stroke, hemolysis, acute respiratory disorder (ARDS), and the like.

“Diseases resulting from oxidative stress” refers to any disease that involves the generation of free radicals or radical compounds, such as, for example, atherogenesis, atheromatosis, arteriosclerosis, atherosclerosis, vascular hypertrophy associated with hypertension, hyperlipoproteinaemia, normal vascular degeneration through aging, parathyroidal reactive hyperplasia, renal disease (e.g., acute or chronic), neoplastic diseases, inflammatory diseases, neurological and acute bronchopulmonary disease, tumorigenesis, ischemia-reperfusion syndrome, arthritis, sepsis, cognitive dysfunction, endotoxic shock, endotoxin-induced organ failure, and the like.

“Endothelial dysfunction” refers to the impaired ability in any physiological processes carried out by the endothelium, in particular, production of nitric oxide regardless of cause. It may be evaluated by, such as, for example, invasive techniques, such as, for example, coronary artery reactivity to acetylcholine or methacholine, and the like, or by noninvasive techniques, such as, for example, blood flow measurements, brachial artery flow dilation using cuff occlusion of the arm above or below the elbow, brachial artery ultrasonography, imaging techniques, measurement of circulating biomarkers, such as, asymmetric dimethylarginine (ADMA), and the like. For the latter measurement the endothelial-dependent flow-mediated dilation will be lower in patients diagnosed with an endothelial dysfunction.

“Methods for treating endothelial dysfunction” include, but are not limited to, treatment prior to the onset/diagnosis of a disease that is caused by or could result from endothelial dysfunction, such as, for example, atherosclerosis, hypertension, diabetes, heart failure, and the like.

“Methods for treating diseases caused by endothelial dysfunction” include, but are not limited to, the treatment of any disease resulting from the dysfunction of the endothelium, such as, for example, arteriosclerosis, heart failure, hypertension, cardiovascular diseases, cerebrovascular diseases, renovascular diseases, mesenteric vascular diseases, pulmonary vascular diseases, ocular vascular diseases, peripheral vascular diseases, peripheral ischemic diseases, and the like.

“Renovascular diseases” refers to any disease or dysfunction of the renal system including, but not limited to, renal failure (e.g., acute or chronic), renal insufficiency, nephrotic edema, acute glomerulonephritis, oliguric renal failure, renal deterioration associated with severe hypertension, unilateral perechymal renal disease, polycystic kidney disease, chronic pyelonephritis, renal diseases associated with renal insufficiency, complications associated with dialysis or renal transplantation, renovascular hypertension, nephropathy, glomerulonephritis, scleroderma, glomerular sclerosis, renal artery stenosis, AIDS-associated nephropathy, immune-mediated renal disease, atheroembolic renal disease, pre-renal azotemia, and the like.

“Angiotensin converting enzyme (ACE) inhibitor” refers to compounds that inhibit an enzyme which catalyzes the conversion of angiotensin I to angiotensin II. ACE inhibitors include, but are not limited to, amino acids and derivatives thereof, peptides, including di- and tri-peptides, and antibodies to ACE which intervene in the renin-angiotensin system by inhibiting the activity of ACE thereby reducing or eliminating the formation of the pressor substance angiotensin II.

“Angiotensin II antagonists” refers to compounds which interfere with the function, synthesis or catabolism of angiotensin II. Angiotensin II antagonists include peptide compounds and non-peptide compounds, including, but not limited to, angiotensin II antagonists, angiotensin II receptor antagonists, agents that activate the catabolism of angiotensin II, and agents that prevent the synthesis of angiotensin I from angiotensin II. The renin-angiotensin system is involved in the regulation of hemodynamics and water and electrolyte balance. Factors that lower blood volume, renal perfusion pressure, or the concentration of sodium in plasma tend to activate the system, while factors that increase these parameters tend to suppress its function.

“Diuretic compound” refers to and includes any compound or agent that increases the amount of urine excreted by a patient.

“Excipient” or “carriers” refers to materials suitable for compound administration and include any such material known in the art such as, for example, any liquid, gel, solvent, solubilizer, or the like, which is non-toxic and which does not interact with any components of the composition in a deleterious manner.

“Formulation” denotes a system for administering an effective amount of the active ingredients to a patient in need of therapy. The formulation may be administered once-daily, twice-daily or thrice-daily. Alternatively, two of the same formulations may be administered once-daily, twice-daily or thrice-daily (e.g., two doses (i.e. tablets, capsules, and the like) may be administered twice-daily or three doses (i.e. tablets, capsules and the like) may be administered thrice-daily).

“qd” refers to the administration of a formulation once during a 24 hour period.

“bid” refers to the administration of a formulation twice during a 24 hour period.

“tid” refers to the administration of a formulation three times during a 24 hour period.

“Immediate release” (“IR”) formulation refers to a formulation the releases one or more of the active ingredients within about 2 minutes; within about 5 minutes; within about 10 minutes; within about 15 minutes; within about 20 minutes; within about 25 minutes; within about 30 minutes; within about 45 minutes; within about 60 minutes; within about 90 minutes; within about 120 minutes; after administration of the formulation. The immediate release profile of the formulation can be determined by the dissolution profile of the active ingredients in vitro. In one embodiment, the immediate release formulations deliver the active ingredients within a period of time as described above so that the blood levels of the active compound(s) are obtained within a desirable range. The immediate release formulations can be prepared using any conventional method known to one skilled in the art to obtain the desired release characteristics.

“Sustained release” (“SR”) formulation refers to a formulation that releases one or more of the active ingredients over a period of time ranging from about 2 minutes to about 8 hours; from about 2 minutes to about 6 hours; from about 2 minutes to about 4 hours; from about 5 minutes to about 8 hours; from about 5 minutes to about 6 hours; from about 5 minutes to about 4 hours; from about 10 minutes to about 8 hours; from about 10 minutes to about 6 hours; from about 10 minutes to about 4 hours; from about 15 minutes to about 8 hours; from about 15 minutes to about 6 hours; from about 15 minutes to about 4 hours; from about 20 minutes to about 8 hours; from about 20 minutes to about 6 hours; from about 20 minutes to about 4 hours; from about 25 minutes to about 8 hours; from about 25 minutes to about 6 hours; from about 25 minutes to about 4 hours; from about 30 minutes to about 8 hours; from about 30 minutes to about 6 hours; from about 30 minutes to about 4 hours; from about 45 minutes to about 8 hours; from about 45 minutes to about 6 hours; from about 45 minutes to about 4 hours; from about 60 minutes to about 8 hours; from about 60 minutes to about 6 hours; from about 60 minutes to about 4 hours; from about 90 minutes to about 8 hours; from about 90 minutes to about 6 hours; from about 90 minutes to about 4 hours; from about 120 minutes to about 8 hours; or from about 120 minutes to about 6 hours; from about 120 minutes to about 4 hours; after administration of the formulation. The sustained release profile of the formulation can be determined by the dissolution profile of the active ingredients in vitro. The sustained release formulations can be zero-order release formulations. Zero-order release denotes formulations that deliver the active ingredients at a uniform rate to dampen the peaks and valleys observed in non-zero order method of drug delivery. Zero-order release formulations may also be referred to as controlled release. In one embodiment, the sustained release formulations deliver the active ingredients over a period of time as described above so that the blood levels of the active compound(s) are maintained within a desirable range. The sustained release formulations can be prepared using any conventional method known to one skilled in the art to obtain the desired release characteristics.

“Variable release” (“VR”) refers to a formulation that comprises at least one immediate release form (the IR release profile of which is described above) and at least one sustained release form (the SR release profile of which is described above). In one embodiment, the variable release formulations deliver the active ingredients within a period of time so that the blood levels of the active compound(s) are maintained within a desirable range.

“Delayed release” refers to the release of one or more of the active ingredients after about 2 to 10 hours; after about 2 to 8 hours; after about 2 to 6 hours; after about 2 to 4 hours; 4 to 10 hours; after about 4 to 8 hours; after about 4 to 6 hours; after the administration of the formulation. The release of the active ingredients in a delayed release form can be, for example, as an immediate release, as a sustained release, and/or as a variable release. The delayed release formulations can be prepared using any conventional method known to one skilled in the art to obtain the desired release characteristics.

“Pulsed release” refers (i) to the immediate release of one or more active compounds, (ii) followed by one or more delayed releases (e.g., as IR, SR and/or VR) of one or more of the active ingredients. By incorporating both an immediate release and one or more delayed releases of the active ingredients, the pulsed release formulation can mimic a multiple dosing profile without repeated dosing, i.e., multiple dosing can be achieved by administering a single formulation in one day. For example, the pulsed release formulation can provide a bid dosing profile when the formulation contains an immediate release form and a single delayed release form (e.g., the formulation provides for an immediate release of the active ingredient(s), followed by a period of time where no active ingredient(s) are released, followed by an immediate, sustained release and/or variable release of the active ingredient(s)). Alternatively, the pulsed release formulation can provide a tid dosing profile when the formulation contains an immediate release form and two delayed release forms (e.g., the formulation provides for an immediate release of the active ingredient(s), followed by a period of time where no active ingredient(s) are released, followed by an immediate, sustained release and/or variable release of the active ingredient(s), optionally followed by a period of time where no active ingredient(s) are released [i.e., in the case where the prior delayed release form was only an immediate release form], followed by another immediate, sustained release and/or variable release of the active ingredients). The pulsed release formulations can be prepared using any conventional method known to one skilled in the art to obtain the desired release characteristics.

“Peak exhaled nitrogen oxides (NO) levels” refers to the NO measured in exhaled breath using any NO analyzer known to one skilled in the art, such as, for example, a chemoluminiscence analyzer, a colormetric detector, and the like. The exhaled peak NO level is the difference between the NO levels obtained before and after administration of a nitric oxide donating compound (i.e. ISDN, ISMN) to the same person.

The term “hydralazine compound” refers to a compound having the formula:

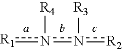

or a pharmaceutically acceptable salt thereof; wherein a, b and c are each independently a single or a double bond; R₁ and R₂ are each independently a hydrogen, an alkyl, an ester or a heterocyclic ring; R₃ and R₄ are each independently a lone pair of electrons or a hydrogen, with the proviso that at least one of R₁, R₂, R₃ and R₄ is not a hydrogen. Exemplary hydralazine compounds include budralazine, cadralazine, dihydralazine, endralazine, hydralazine, pildralazine, todralazine and the like. In one embodiment, the hydralazine pharmaceutically acceptable salt of the hydralazine compound is hydralazine hydrochloride. The plasma concentration of hydralazine resulting from the administration of hydralazine hydrochloride can be determined by any analytical technique known in the art, such as for example, HPLC, gas chromatography, mass spectrometry, and the like.

The term “hydrazone compound” refers to a compound with the structure R₂N—N(H)═CR₂, wherein R₂ is as defined herein. Hydrazone compounds are typically formed by the reaction of the hydrazino group in the hydralazine compound with aldehydes and ketones in the formulation. The chemical structure of the hydrazone compound can be determined using any analytical methods known to one skilled in the art, including, but not limited to, high pressure liquid chromatography, ¹H NMR, ¹³C NMR, mass spectroscopy, and the like.

“Water content of the formulation” refers to the total amount of water present in the final formulation. The water content of the formulation can be determined using any analytical methods known to one skilled in the art, including, but not limited to, loss on drying, Karl Fisher titration, thermoanalytical analysis, and the like.

The terms “active ingredient” and “active ingredients” refer to a hydralazine compound (e.g. hydralazine hydrochloride) and, optionally, at least one of ISDN and ISMN. In one embodiment, the “active ingredient(s)” is hydralazine hydrochloride. In another embodiment, the “active ingredient(s)” is hydralazine hydrochloride and ISDN. Whether the singular or plural of the word “active ingredient(s)” is used, it is intended to include at least one compound, i.e., i.e., a hydralazine compound; or two compounds, i.e., a hydralazine compound and at least one of ISDN and ISMN;

The term “ISMN” (isosorbide mononitrate) is intended to include isosorbide 5-mononitrate and isosorbide 2-mononitrate unless specified otherwise.

The term “at least one of ISDN and ISMN” refers to (i) ISDN, (ii) ISMN, or (iii) ISDN and ISMN. ISDN is the one embodiment.

Isosorbide dinitrate (ISDN) is commercially available, for example, under the trade names DILATRATE®-SR (Schwarz Pharma, Milwaukee, Wis.); ISORDIL® and ISORDILR TITRADOSE® (Wyeth Laboratories Inc., Philadelphia, Pa.); and SORBITRATE® (Zeneca Pharmaceuticals, Wilmington, Del.). Diluted isosorbide dinitrate (1,4,3,6-dianhydro-D-glucitol-2,5-dinitrate), USP, is a white to off-white powder that has a melting point of 70° C. and has an optical rotation of +1350 (3 mg/mL, ethanol). It is freely soluble in organic solvents such as ethanol, ether and chloroform, but is sparingly soluble in water. The major active metabolites of ISDN are isosorbide 5-mononitrate and isosorbide 2-mononitrate. Isosorbide 5-mononitrate is reported as having a longer half life in vivo. As isosorbide dinitrate is explosive in nature it is generally stored and shipped in a carrier such as, for example, lactose, sorbitol, dextrose, mannitol, cellulose, microcrystalline cellulose, and the like. The plasma concentration of the metabolites resulting from the administration of ISDN can be determined by any analytical technique known in the art, such as for example, HPLC, gas chromatography, mass spectrometly, and the like.

Isosorbide mononitrate (ISMN) is commercially available, for example, under the trade names IMDUR® (A. B. Astra, Sweden); MONOKET® (Schwarz Pharma, Milwaukee, Wis.); and ISMO® (Wyeth-Ayerst Company, Philadelphia, Pa.). As isosorbide mononitrate is explosive in nature it is generally stored and shipped in a carrier such as, for example, lactose, sorbitol, dextrose, mannitol, celluloase, microcrystalline cellulose, and the like.

“Pharmaceutically acceptable salt” refers to, for example, alkali metal salts and addition salts of free acids or free bases of the corresponding compound. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitrous (nitrite salt), nitric (nitrate salt), carbonic, sulfuric, phosphoric acid, and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic; fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

“Alkyl” refers to a lower alkyl group, a substituted lower alkyl group, a haloalkyl group, a hydroxyalkyl group, an alkenyl group, a substituted alkenyl group, an alkynyl group, a bridged cycloalkyl group, a cycloalkyl group or a heterocyclic ring, as defined herein. An alkyl group may also comprise one or more radical species, such as, for example a cycloalkylalkyl group or a heterocyclicalkyl group. “Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Substituted lower alkyl” refers to a lower alkyl group, as defined herein, wherein one or more of the hydrogen atoms have been replaced with one or more R¹⁰⁰ groups, wherein each R¹⁰⁰ is independently a hydroxy, an ester, an amidyl, an oxo, a carboxyl, a carboxamido, a halo, a cyano, a nitrate or an amino group, as defined herein.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like.

“Alkenyl” refers to a branched or straight chain C₂-C₁₀ hydrocarbon (preferably a C₂-C₈ hydrocarbon, more preferably a C₂-C₆ hydrocarbon) that can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like. “Lower alkenyl” refers to a branched or straight chain C₂-C₄ hydrocarbon that can comprise one or two carbon-carbon double bonds. “Substituted alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Alkynyl” refers to an unsaturated acyclic C₂-C₁₀ hydrocarbon (preferably a C₂-C₈ hydrocarbon, more preferably a C₂-C₆ hydrocarbon) that can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo(3.3.0)octane, 7-oxabicyclo(2.2.1)heptyl, 8-azabicyclo(3,2,1)oct-2-enyl and the like. “Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo, alkylsulfinyl, and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic compounds” refer to mono- and polycyclic compounds comprising at least one aryl or heterocyclic ring. “Heterocyclic ring or group” refers to a saturated or unsaturated cyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur maybe in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylthio, aryloxy, arylthio, arylalkyl, hydroxy, oxo, thial, halo, carboxyl, carboxylic ester, alkylcarboxylic acid, alkylcarboxylic ester, aryl, arylcarboxylic acid, arylcarboxylic ester, amidyl, ester, alkylcarbonyl, arylcarbonyl, alkylsulfinyl, carboxamido, alkylcarboxamido, arylcarboxamido, sulfonic acid, sulfonic ester, sulfonamide nitrate and nitro. Exemplary heterocyclic groups include pyrrolyl, furyl, thienyl, 3-pyrrolinyl, 4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrahydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl, 2,6-dioxabicyclo(3.3.0)octane, and the like.

“Aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl, and the like. Aryl groups (including bicyclic aryl groups) can be unsubstituted or substituted with one, two or three substituents independently selected from alkyl, alkoxy, alkylthio, amino, alkylamino, dialkylamino, arylamino, diarylamino, alkylarylamino, halo, cyano, alkylsulfinyl, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbornyl, sulfonic acid, sulfonic ester, sulfonamido and nitro. Exemplary substituted aryl groups include tetrafluorophenyl, pentafluorophenyl, sulfonamide, alkylsulfonyl, arylsulfonyl, and the likc.

“Hydroxy” refers to —OH. “Hydroxyalkyl” refers to a hydroxy group, as defined herein, appended to an alkyl group, as defined herein.

“Alkylcarbonyl” refers to R₅₂—C(O)—, wherein R₅₂ is an alkyl group, as defined herein. “Arylcarbonyl” refers to R₅₅—C(O)—, wherein R₅₅ is an aryl group, as defined herein.

“Ester” refers to R₅₁C(O)O— wherein R₅₁ is a hydrogen atom, an alkyl group, an aryl group, an alkylaryl group, or an arylheterocyclic ring, as defined herein.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetra-hydroquinoline, and the like.

“Hydrazino” refers to H₂N—N(H)—.

Hydralazine compounds (e.g. hydralazine hydrochloride) readily undergoes reactions with aldehydes and ketones due to its highly reactive hydrazino group resulting in the formation of hydrazones that can undergo further reaction and/or degradation. Hydralazine hydrochloride also undergoes several pharmaceutically undesirable reactions such as chelation with metal ions, oxidation, and pH-dependent decomposition. These reactions often cause discoloration of the compositions comprising hydralazine compounds. One embodiment provides stable, solid dosage hydralazine composition that could be manufactured easily and stored for periods of time without significant degradation of the hydralazine compound. In one embodiment, the hydralazine compound is hydralazine hydrochloride. In these embodiments, the total amount of hydrazone compounds is less than about 0.001% to about 0.1% of the total weight of the formulation The formulations further comprise at least one chelating agent and at least one acidic agent. In another embodiment, the hydralazine formulations are essentially free of metal ions. In yet another embodiment, the hydralazine formulations have a water content of about 1% to about 3% based on the total weight of the formulation at the time of manufacture. The invention also provides methods for making such formulations.

The invention also provides solid dosage formulations comprising at least one hydralazine compound or a pharmaceutically acceptable salt thereof, and at least one nitric oxide donor compound or a pharmaceutically acceptable salt thereof and at least one excipient or carrier, wherein the formulations have less than about 0.001% to about 0.1% of a hydrazone compound based on the total weight of the formulation. In one embodiment the hydralazine compound is hydralazine hydrochloride. In another embodiment, the nitric oxide donor compound is ISDN or ISMN. In yet another embodiment, the formulations comprise a hydralazine compound and at least one of isosorbide dinitrate and isosorbide mononitrate. In another embodiment, the solid dosage formulations comprise hydralazine hydrochloride and isosorbide dinitrate. In these embodiments, the formulations comprising at least one hydralazine compound or a pharmaceutically acceptable salt thereof does not react with the at least one excipient or carrier to form hydrazone compounds. In these embodiments, the total amount of hydrazone compounds is less than about 0.001% to about 0.1% of the total weight of the formulation. The formulations further comprise at least one chelating agent and at least one acidic agent. In another embodiment, the hydralazine formulations are essentially free of metal ions. In yet another embodiment, the hydralazine formulations have a water content of about 1% to about 3% based on the total weight of the formulation at the time of manufacture. The invention also provides methods for making such formulations.

Suitable excipients or carriers that will form hydrazone compounds in a total amount that is less than about 0.001% to about 0.1% of the total weight of the formulation by the reaction of the hydrazino group in the hydralazine with aldehydes and ketones in the excipient or carrier, include, but are not limited to, mannitol USP; sorbitol; microcrystalline cellulose NF; dibasic calcium phosphate dihydrate NF; monobasic calcium sulfate monohydrate; calcium sulfate dihydrate NF; Celutab™; inositol; hydrolyzed cereal solids such as the Maltron™ and Mor-Rex™; amylose; Rexce™; powdered cellulose (e.g., Elcema™); calcium carbonate; glycine; bentonite; polyvinylpyrrolidone; acacia; tragacanth; gelatin; alginic acid and salts of alginic acid; magnesium aluminum silicate; polyethylene glycol; guar gum; polysaccharide acids; bentonites; polyvinylpyrrolidone (povidone); polymethacrylates; pregelatinized starch (such as National™ 1511 and Starch 1500); silicon dioxide, titanium dioxide, cellulosic carrier materials such as cellulose materials or a cellulose derivative such as, for example, purified cellulose; microcrystalline cellulose, alkyl celluloses and their derivatives and salts, methylcellulose, sodium carboxymethylcellulose, carboxymethylcellulose, croscarmellose sodium, hydroxypropyl cellulose, hydroxypropyl methylcellulose, hydroxypropyl methylcellulose phthalate, hydroxypropyl methylcellulose acetate succinate, ethyl cellulose, cellulose acetate phthalate, phthalate ethyl cellulose, and the like, and combinations of two or more thereof. In one embodiment the carrier material is a cellulosic material selected from the group consisting of C₁-C₆ alkyl celluloses and their derivatives and salts, hydroxyalkyl alkylcelluloses and their derivatives and salts, hydroxyl(C₂-C₄ alkyl) (C₁-C₄ alkyl)celluloses and their derivatives and salts. The excipients or carriers exhibit suitable compressibility and pre-compression flow properties. In another embodiment, the excipient or carrier includes, but is not limited to, non-reducing sugars, such as, for example, microcrystalline cellulose (e.g. Avicel® PH 101), cellulose, mannitol or sorbitol either individually or as combinations of two or more thereof. In one embodiment, the excipient or carrier is microcrystalline cellulose. In another embodiment the excipient or carrier is mannitol.

In another embodiment, the solid dosage formulations comprising a hydralazine compound or a pharmaceutically acceptable salt thereof (e.g., hydralazine hydrochloride) and, optionally, at least one of isosorbide dinitrate and isosorbide mononitrate (e.g., ISDN) contain at least one chelating agent, such as, for example, ethylenediamine tetraacetic acid, ethylene glycol tetraacetic acid, 1,2-bis(o-aminophenoxy)ethane-N,N,N′,N′-tetraacetic acid, nitrilotriacetic acid, and pharmaceutically acceptable salts thereof, such as, for example, sodium salt, calcium salt, magnesium salt, and the like, to stabilize the hydralazine hydrochloride from degradation by chelation of the metal ions and thereby resulting in a formulation that is essentially free of metal ions. In one embodiment the chelating agent is present at about 0.01% to about 0.05% of the total weight of the formulation. In another embodiment the chelating agent is present at about 0.02% to about 0.04% of the total weight of the formulation. In yet another embodiment the chelating agent is present at about 0.02% of the total weight of the formulation. In one embodiment the chelating agent is ethylenediamine tetraacetic acid, ethylenediamine tetraacetic acid disodium salt, or a combination thereof.

In another embodiment, the solid dosage formulations comprising a hydralazine compound or a pharmaceutically acceptable salt thereof (e.g., hydralazine hydrochloride) and, optionally, at least one of isosorbide dinitrate and isosorbide mononitrate (e.g., ISDN) contain at least one acidic agent, such as, for example, citric acid, fumaric acid, malic acid, ascorbic acid, tartaric acid, lactic acid, and the like, to stabilize the hydralazine hydrochloride by maintaining the pH of the overall formulation below about pH 4.0. In these embodiments the acidic agent is present at about 0.01% to about 0.5% of the total weight of the formulation. In another embodiment, the acidic agent is present at about 0.05% to about 0.3% of the total weight of the formulation. In another embodiment, the acidic agent is present at about 0.1% to about 0.2% of the total weight of the formulation. In yet another embodiment the acidic agent is present at about 0.1% of the total weight of the formulation. In one embodiment the acidic agent is citric acid, fumaric acid, ascorbic acid or a combination thereof.

In another embodiment, the solid dosage formulations comprising a hydralazine compound or a pharmaceutically acceptable salt thereof (e.g., hydralazine hydrochloride) and, optionally, at least one of isosorbide dinitrate and isosorbide mononitrate (e.g., ISDN) have a water content of about 1% to about 3% based on the total weight of the formulation as determined at the time of manufacturing the final formulation. In another embodiment, the formulations have a water content of about 2% based on the total weight of the formulation as determined at the time of manufacturing the final formulation.

In one embodiment, the solid dosage formulations comprising a hydralazine compound or a pharmaceutically acceptable salt thereof (e.g., hydralazine hydrochloride) and, optionally, at least one of isosorbide dinitrate and isosorbide mononitrate (e.g., ISDN) is in a solid dosage formulation for oral administration. The solid dosage formulation can be any known in the art. Exemplary solid dosage formulations include beads, microspheres, pellets, tablets, granules, capsules, crystals, lozenges, powders, gels, caplets, and the like. In one embodiment the solid dosage formulation is a tablet. In another embodiment the solid dosage formulation is a capsule. In yet another embodiment the solid dosage formulation is a granule. In these solid dosage formulations the hydralazine compound (e.g., hydralazine hydrochloride), and optional nitric oxide donor compound (e.g. ISDN and/or ISMN) can be in an immediate release form, in a sustained release form, in a delayed release form, in a variable release form, in a pulsed release form or in a combination thereof. In these embodiments, the immediate release form, the sustained release form, the delayed release form, the variable release form and the pulsed release form can be any immediate release formulation, any sustained release formulation, any delayed release formulation, any variable release formulation or any pulsed release formulation known to one skilled in the art to obtain the desired release characteristics, including, but not limited to, those described herein.

In one embodiment, the solid dosage formulations comprising a hydralazine compound or a pharmaceutically acceptable salt thereof (e.g., hydralazine hydrochloride) and, optionally, at least one of isosorbide dinitrate and isosorbide mononitrate (e.g., ISDN) is as an injectable formulation. The injectable formulation can be any injectable formulation known to one skilled in the art to obtain the desired release characteristics, including, but not limited to, those described herein.

In one embodiment, the immediate release, sustained release, delayed release, variable release and/or pulsed release solid dosage formulations comprise hydralazine hydrochloride in an amount of about 20 milligrams to about 400 milligrams. In other embodiments, the immediate release, sustained release, delayed release, variable release and/or pulsed release formulations comprise hydralazine hydrochloride in an amount of about 30 milligrams to about 300 milligrams. In other embodiments, the immediate release, sustained release, delayed release, variable release and/or pulsed release formulations comprise hydralazine hydrochloride in an amount of about 37.5 milligrams to about 225 milligrams. In other embodiments, the immediate release formulations comprise hydralazine hydrochloride in an amount of about 25 milligrams to about 100 milligrams. In other embodiments, the immediate release formulations comprise hydralazine hydrochloride in an amount of about 25 milligrams. In other embodiments, the immediate release comprise hydralazine hydrochloride in an amount of about 37.5 milligrams. In other embodiments, the immediate release formulations comprise hydralazine hydrochloride in an amount of about 50 milligrams. In other embodiments, the immediate release formulations comprise hydralazine hydrochloride in an amount of about 75 milligrams. In other embodiments, the immediate release formulations comprise hydralazine hydrochloride in an amount of about 100 milligrams. In these embodiments, the total amount of hydrazone compounds in the formulation is less than about 0.001% to about 0.1% of the total weight of the formulation.

In one embodiment, the immediate release, sustained release, delayed release, variable release and/or pulsed release solid dosage formulations comprise (1) hydralazine hydrochloride in an amount of about 20 milligrams to about 400 milligrams; and (2) isosorbide dinitrate in an amount of about 10 milligrams to about 200 milligrams and/or isosorbide mononitrate in an amount of about 5 milligrams to about 120 milligrams. In other embodiments, the immediate release, sustained release, delayed release, variable release and/or pulsed release formulations comprise (1) hydralazine hydrochloride in an amount of about 30 milligrams to about 300 milligrams; and (2) isosorbide dinitrate in an amount of about 20 milligrams to about 160 milligrams and/or isosorbide mononitrate in an amount of about 15 milligrams to about 100 milligrams. In other embodiments, the immediate release, sustained release, delayed release, variable release and/or pulsed release formulations comprise (1) hydralazine hydrochloride in an amount of about 37.5 milligrams to about 225 milligrams; and (2) isosorbide dinitrate in an amount of about 20 milligrams to about 120 milligrams and/or isosorbide mononitrate in an amount of about 10 milligrams to about 60 milligrams. In these embodiments, the total amount of hydrazone compounds in the formulation is less than about 0.001% to about 0.1% of the total weight of the formulation.

In other embodiments, the sustained release, delayed release, variable release and/or pulsed release solid dosage formulations comprise hydralazine hydrochloride in an amount of about 112.5 milligrams; and isosorbide dinitrate in an amount of about 60 milligrams and/or isosorbide mononitrate in an amount of about 30 milligrams. The sustained release, delayed release, variable release and/or pulsed release, when orally administered to a human patient in need thereof, produce a blood plasma concentration from about 100 ng/mL to about 800 ng/mL isosorbide 5-mononitrate; from about 150 ng/mL to about 600 ng/mL isosorbide 5-mononitrate; or from about 200 ng/mL to about 400 ng/mL isosorbide 5-mononitrate; and/or produce a blood plasma concentration from about 5 ng/mL to about 100 ng/mL hydralazine; or from about 20 ng/mL to about 80 ng/mL hydralazine; and/or result in the production of from about 2 ppb to about 60 ppb of peak exhaled nitric oxide levels; or from about 5 ppb to about 40 ppb of peak exhaled nitric oxide levels. In these embodiments, the total amount of hydrazone compounds in the formulation is less than about 0.001% to about 0.1% of the total weight of the formulation.

In other embodiments, the immediate release, sustained release, delayed release, variable release and/or pulsed release solid dosage formulations comprise hydralazine hydrochloride in an amount of about 56.25 milligrams; and isosorbide dinitrate in an amount of about 30 milligrams and/or isosorbide mononitrate in an amount of about 15 milligrams. The immediate release, sustained release, delayed release, variable release and/or pulsed release formulation when orally administered to a human patient in need thereof, produce a blood plasma concentration from about 50 ng/mL to about 400 ng/mL isosorbide 5-mononitrate; from about 75 ng/mL to about 300 ng/mL isosorbide 5-mononitrate; or from about 100 ng/mL to about 200 ng/mL isosorbide 5-mononitrate; and produce a blood plasma concentration from about 2.5 ng/mL to about 50 ng/mL hydralazine; or from about 10 ng/mL to about 40 ng/mL hydralazine; and/or produce about 1 ppb to about 30 ppb of peak exhaled nitric oxide levels; or from about 2.5 ppb to about 20 ppb of peak exhaled nitric oxide levels. In these embodiments, the total amount of hydrazone compounds in the formulation is less than about 0.001% to about 0.1% of the total weight of the formulation.

In one embodiment, the sustained release, delayed release, variable release and/or pulsed release solid dosage formulations comprise hydralazine hydrochloride in an amount of about 112.5 milligrams; and isosorbide dinitrate in an amount of about 60 milligrams are administered orally bid. In another embodiment, sustained release, delayed release, variable release and/or pulsed release formulations comprise hydralazine hydrochloride in an amount of about 56.25 milligrams; and isosorbide dinitrate in an amount of about 30 milligrams are administered orally bid. In another embodiment, two doses of the sustained release, delayed release, variable release and/or pulsed release formulations comprise hydralazine hydrochloride in an amount of about 56.25 milligrams; and isosorbide dinitrate in an amount of about 30 milligrams are administered orally bid at about the same time. “About the same time” includes administering the at least two doses of the variable release formulation simultaneously, sequentially or at the same time.

In another embodiment, the pulsed release solid dosage formulations comprise about 225 mg hydralazine hydrochloride and about 120 mg isosorbide dinitrate and/or about 60 milligrams isosorbide mononitrate. The hydralazine hydrochloride, isosorbide dinitrate and/or isosorbide mononitrate in the pulsed release formulations are in an immediate release form and a delayed release form. For example, the pulsed release formulation comprise about 75 mg hydralazine hydrochloride, about 40 milligrams isosorbide dinitrate and/or about 20 milligrams isosorbide mononitrate in an immediate release form; about 75 mg hydralazine hydrochloride, about 40 milligrams isosorbide dinitrate and/or about 20 milligrams isosorbide mononitrate in the first delayed release form; and about 75 mg hydralazine hydrochloride, about 40 milligrams isosorbide dinitrate and/or about 20 milligrams isosorbide mononitrate in the second delayed release form. This pulsed release formulation can provide a tid dosing profile by the single administration of the formulation. The pulsed release formulation, when orally administered to a human patient in need thereof, produces a blood plasma concentration from about 100 ng/mL to about 800 ng/mL isosorbide 5-mononitrate; from about 150 ng/mL to about 600 ng/mL isosorbide 5-mononitrate; or from about 200 ng/mL to about 400 ng/mL isosorbide 5-mononitrate; and/or produces a blood plasma concentration from about 5 ng/mL to about 100 ng/mL hydralazine; or from about 20 ng/mL to about 80 ng/mL hydralazine; and/or produces about 2 ppb to about 60 ppb of peak exhaled nitric oxide levels; or about 5 ppb to about 40 ppb of peak exhaled nitric oxide levels. In these embodiments, the total amount of hydrazone compounds in the formulation is less than about 0.001% to about 0.1% of the total weight of the formulation.

In another embodiment, the pulsed release solid dosage formulations comprise about 112.5 mg hydralazine hydrochloride and about 60 mg isosorbide dinitrate and/or about 60 milligrams isosorbide mononitrate. The hydralazine hydrochloride, isosorbide dinitrate and/or isosorbide mononitrate in the pulsed release formulations comprise an immediate release form and a delayed release form. For example, the pulsed release formulations comprise about 37.5 mg hydralazine hydrochloride, about 20 milligrams isosorbide dinitrate and/or about 10 milligrams isosorbide mononitrate in an immediate release form; about 37.5 mg hydralazine hydrochloride, about 20 milligrams isosorbide dinitrate and/or about 10 milligrams isosorbide mononitrate in the first delayed release form; and about 37.5 mg hydralazine hydrochloride, about 20 milligrams isosorbide dinitrate and/or about 10 milligrams isosorbide mononitrate in the second delayed release form. This pulsed release formulation can provide a tid dosing profile by the single administration of the formulation. The pulsed release formulation, when orally administered to a human patient in need thereof, produces a blood plasma concentration of about 50 ng/mL to about 300 ng/mL of isosorbide 5-mononitrate, or from about 100 ng/mL to about 200 ng/mL of isosorbide 5-mononitrate; and/or produces a blood plasma concentration from about 2.5 ng/mL to about 50 ng/mL hydralazine; or about 10 ng/mL to about 40 ng/mL hydralazine; and/or produces about 1 ppb to about 30 ppb of peak exhaled nitric oxide levels; or about 2.5 ppb to about 20 ppb of peak exhaled nitric oxide levels. In these embodiments, the total amount of hydrazone compounds in the formulation is less than about 0.001% to about 0.1% of the total weight of the formulation.

In one embodiment, the pulsed release solid dosage formulations comprise about 225 mg hydralazine hydrochloride and about 120 mg isosorbide dinitrate are administered orally qd. In another embodiment, the pulsed release formulations comprise about 112.5 mg hydralazine hydrochloride and about 60 mg isosorbide dinitrate are administered orally qd.

In addition to the excipients or carriers, the solid dosage formulations may further comprise one or more pharmaceutically acceptable binders, disintegrants, lubricants and/or glidants, adhesives, surfactants, wetting agents, anti-adherent agents, water insoluble polymers, plasticizers, enteric coating polymers, granulating aids, colorants, flavorants, and/or other carrier materials, that are conventional in the pharmaceutical art.

Suitable binders include, but are not limited to, pre-gelatinized starch, gelatin, sugars (including sucrose, glucose, dextrose, lactose and sorbitol), polyethylene glycol, waxes, natural and synthetic gums, such as, for example, acacia, tragacanth, sodium alginate, cellulose, including hydrorroxypropylmethylcellulose, hydroxypropylcellulose, ethylcellulose, veegum, and the like; synthetic polymers, such as, for example, acrylic acid and methacrylic acid copolymers, methacrylic acid copolymers, methyl methacrylate copolymers, aminoalkyl methacrylate copolymers, polyacrylic acid/polymethacrylic acid, polyvinylpyrrolidone, and the like. Some of the materials which are suitable as binders can also be used as matrix-forming materials, such as, for example, hydroxypropyl methyl cellulose, ethyl cellulose, microcrystalline cellulose, and the like.

Suitable disintegrants include, but are not limited to, starches; sodium starch glycolate; clays (such as Veegum™ HV); celluloses (such as purified cellulose, methylcellulose and sodium carboxymethylcellulose, sodium carboxymethylcellulose, hydroxypropyl cellulose); alginates; pregelatinized corn starches (such as National™ 1551 and National™ 1550); crospovidone USP NF; gums (such as agar, guar, locust bean, Karaya™, pectin, and tragacanth) cross carmellose sodium, alginates, and the like. Disintegrants can be added at any suitable step during the preparation of the pharmaceutical composition, particularly prior to granulation or during the lubrication step prior to compression. In one embodiment, the disintegrant is sodium starch glycolate.

Suitable lubricants and/or glidants include, but are not limited to, glyceryl behenate (Compritol™ 888); metallic stearates (e.g., magnesium, calcium and sodium stearates); stearic acid; sodium stearyl fumarate, hydrogenated vegetable oils (e.g., Sterotex™); talc; waxes; Stearowet™; boric acid; sodium benzoate and sodium acetate; sodium chloride; DL-Leucine; polyethylene glycols (e.g., Carbowax™ 4000 and Carbowax™ 6000); sodium oleate; sodium benzoate; sodium acetate; sodium lauryl sulfate; sodium stearyl fumarate (Pruv™); magnesium lauryl sulfate; silicon dioxide, titanium dioxide, polyethylene glycol waxes; and the like and combinations of two or more thereof. In one embodiment, the lubricant is magnesium stearate or silicon dioxide and combinations thereof.

Suitable surfactants include, but are not limited to, anionic, cationic, amphoteric or nonionic surface active agents. Suitable anionic surfactants include, but are not limited to, those containing carboxylate, sulfonate and sulfate ions. Examples of anionic surfactants include, but are not limited to, sodium, potassium, ammonium salts of long chain alkyl sulfonates and alkyl aryl sulfonates such as, for example, sodium dodecylbenzene sulfonate, and the like; dialkyl sodium sulfosuccinates, such as, for example, sodium dodecylbenzene sulfonate, and the like; dialkyl sodium sulfosuccinates, such as, for example, sodium bis-(2-ethylthioxyl)-sulfosuccinate, and the like; alkyl sulfates such as, for example sodium lauryl sulfate, and the like. Cationic surfactants include, but are not limited to, quaternary ammonium compounds such as, for example, benzalkonium chloride, benzethonium chloride, cetrimonium b romide, stearyl dimethylbenzyl ammonium chloride, polyoxyethylene, coconut amine, and the like. Examples of nonionic surfactants include, but are not limited to, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, polyoxyethylene hydrogenated tallow amide, and the like. Examples of amphoteric surfactants include, but are not limited to, sodium N-dodecyl-.beta.-alanine, sodium N-lauryl-.beta.-iminodipropionate, myristoamphoacetate, lauryl betaine, lauryl sulfobetaine, and the like.

Suitable wetting agents include, but are not limited to, oleic acid; glyceryl monostearate; sorbitan monooleate; sorbitan monolaurate; triethanolamine oleate; polyoxyethylene sorbitan mono-oleate; polyoxyethylene sorbitan monolaurate; sodium oleate; and sodium lauryl sulfate, and the like and combinations of two or more thereof. Such wetting agents improve the bioavailability of the active ingredients.

Suitable anti-adherents include, but are not limited to, talc, cornstarch, Cab-O—Sil™, Syloid™, DL-Leucine, sodium lauryl sulfate, metallic stearates, and the like and combinations of two or more thereof.

Suitable water insoluble polymers include, but are not limited to, ethylcellulose, hydroxypropylmethyl cellulose, hydroxypropyl cellulose, sodium carboxymethyl cellulose, polyethylene glycol, polyvinylpyrrolidone, polyvinyl alcohol, alginic acid, neutral copolymers based on ethylacrylate and methylmethacrylate, copolymers of acrylic acid and methacrylic acid esters having quarternary ammonium groups, polyvinyl acetate, copolymers of polyvinyl acetate and crotonic acid, cellulose acetate, cellulose acetate methyl carbamate, methylcarbamate, cellulose diacetate, polydiethylaminomethylstyrene, cellulose triacetate, cellulose alkanylate, monoalkenytes, dialkenytes, trialkenytes, monoarolyates, diarolyates, triarolyates, cellulose trivalerate, cellulose trioctanoate, cellulose tripionate, cellulose diesters, cellulose disuccinate, cellulose acetate valerate, cellulose acetaldehyde, dimethylcellulose acetate, cellulose dimethylaminoacetate, and the like, and combinations of two or more thereof. In one embodiment, the water insoluble polymer is ethylcellulose, hydroxypropylmethyl cellulose, hydroxypropyl cellulose, sodium carboxymethyl cellulose, neutral copolymers based on ethylacrylate and methylmethacrylate, copolymers of acrylic acid and methacrylic acid esters having quarternary ammonium groups, polyvinyl acetate, copolymers of polyvinyl acetate and crotonic acid, or a combinations of two or more thereof.

Suitable plasticizers include triacetin, citrate esters, tributyl citrate, triethyl citrate, acetyl tri-nbutyl citrate diethyl phthalate, castor oil, dibutyl sebacate, polyethylene glycols, glycerols, acetylated monoglycerides, monoacetylated glycerides, diacetylated glycerides, diacetylated monoglycerides, castor oil, and combinations of two or more thereof. In one embodiment, the plasticizer is acetylated monoglycerides, monoacetylated glycerides, diacetylated glycerides, diacetylated monoglycerides, or combinations of two or more thereof. In another embodiment, the plasticizer is diacetylated monoglycerides.

Suitable enteric-coating polymers include esters of cellulose and/or its derivatives (e.g., cellulose acetate phthalate, hydroxypropyl methylcellulose phthalate, hydroxypropyl methylcellulose acetate succinate), polyvinyl acetate phthalate, carboxymethylethylcellulose, acrylic acid polymers, methacrylic acid copolymers, methacrylic acid-methacrylate copolymers, shellac and combinations of two or more thereof. These enteric polymers may be used as a dry powder or an aqueous dispersion. Some commercially available enteric polymers that may be used are methacrylic acid copolymers sold under the trademark EUDRAGIT® (L100, S100, L30D) manufactured by Rhom Pharma, CELLACEFATE® (cellulose acetate phthalate) from Eastman Chemical Co., AQUATERIC® (cellulose acetate phthalate aqueous dispersion) from FMC Corp. and AQUOT® (hydroxypropyl methylcellulose acetate succinate aqueous dispersion) from Shin Etsu.

The solid dosage formulations are manufactured by standard manufacturing techniques known in the art, such as, by blending, ball-milling, mixing, stirring, roll-milling, extruding, immersing, spray-drying, press-layering, wet granulation, fluid bed granulation, air-suspension coating, compression, and the like. In one embodiment, the combining of the dry ingredients can be done by blending, wet granulation, fluid-bed granulation, or dry granulation according to methods recognized the art.

In alternative embodiments, the “active ingredient(s)” may further comprise one or more additional compounds. For example, the active ingredient(s) may further comprise an angiotensin converting enzyme inhibitor, a β-adrenergic antagonist, an angiotensin II antagonist, an aldosterone antagonist, a digitalis, a diuretic compound, or a combination of two or more thereof. These active ingredients may be incorporated into the immediate release, sustained release, delayed release, variable release and/or pulsed release formulation in the form of an immediate release formulation, sustained release formulation, delayed release formulation, variable release formulation and/or pulsed release formulation or combinations thereof.

In one embodiment, the immediate release, sustained release, delayed release, variable release and/or pulsed release solid dosage formulations further comprise an angiotensin-converting enzyme inhibitor. Suitable angiotensin-converting enzyme inhibitors (ACE inhibitors) include, but are not limited to, alacepril, benazepril (LOTENSIN®, CIBACEN®), benazeprilat, captopril, ceronapril, cilazapril, delapril, duinapril, enalapril, enalaprilat, fasidotril, fosinopril, fosinoprilat, gemopatrilat, glycopril, idrapril, imidapril, lisinopril, moexipril, moveltipril, naphthopidil, omapatrilat, pentopril, perindopril, perindoprilat, quinapril, quinaprilat, ramipril, ramiprilat, rentipril, saralasin acetate, spirapril, temocapril, trandolapril, trandolaprilat, urapidil, zofenopril, acylmercapto and mercaptoalkanoyl pralines, carboxyalkyl dipeptides, carboxyalkyl dipeptide, phosphinylalkanoyl pralines, registry no. 796406, AVE 7688, BP1.137, CHF 1514, E 4030, ER 3295, FPL-66564, MDL 100240, RL 6134, RL 6207, RL 6893, SA 760, S-5590, Z 13752A, and the like. One skilled in the art will appreciate that the angiotensin-converting enzyme inhibitors may be administered in the form of pharmaceutically acceptable salts, hydrates, acids and/or stereoisomers thereof. Suitable angiotensin-converting enzyme inhibitors are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry.

In some embodiments, the solid dosage formulations further comprise benazepril, captopril, enalapril, fosinopril, lisinopril, moexipril, quinapril, ramipril, trandolapril or trandolaprilat. In one embodiment, the formulations further comprise benazepril (e.g., benazepril hydrochloride) in an amount of about 5 milligrams to about 80 milligrams. In one embodiment, the formulations further comprise captopril in an amount of about 12.5 milligrams to about 450 milligrams. In one embodiment, the formulations further comprise enalapril (e.g., enalapril maleate) in an amount of about 2.5 milligrams to about 40 milligrams. In one embodiment, the formulations further comprise fosinopril (e.g., fosinopril sodium) in an amount of about 5 milligrams to about 60 milligrams. In one embodiment, the formulations further comprise lisinopril in an amount of about 2.5 milligrams to about 75 milligrams. In one embodiment, the formulations further comprise moexipril (e.g., moexipril hydrochloride) in an amount of about 7.5 milligrams to about 45 milligrams. In one embodiment, the formulations further comprise quinapril (e.g., quinapril hydrochloride) in an amount of about 5 milligrams to about 40 milligrams. In one embodiment, the formulations further comprise ramapril hydrochloride in an amount of about 1.25 milligrams to about 40 milligrams. In one embodiment, the formulations further comprise trandolapril in an amount of about 0.5 milligrams to about 4 milligrams. In one embodiment, the formulations further comprise trandolaprilat in an amount of about 0.5 milligrams to about 4 milligrams.

In one embodiment, the solid dosage formulations further comprise a β-adrenergic antagonist. Suitable β-adrenergic antagonists include, but are not limited to, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butofilolol, carazolol, capsinolol, carteolol, carvedilol (COREG®), celiprolol, cetamolol, cindolol, cloranolol, dilevalol, diprafenone, epanolol, ersentilide, esmolol, esprolol, hydroxalol, indenolol, labetalol, landiolol, laniolol, levobunolol, mepindolol, methylpranol, metindol, metipranolol, metrizoranolol, metoprolol, moprolol, nadolol, nadoxolol, nebivolol, nifenalol, nipradilol, oxprenolol, penbutolol, pindolol, practolol, pronethalol, propranolol, sotalol, sotalolnadolol, sulfinalol, taliprolol, talinolol, tertatolol, tilisolol, timolol, toliprolol, tomalolol, trimepranol, xamoterol, xibenolol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl) phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide, Acc 9369, AMO-140, BIB-16S, CP-331684, Fr-172516, ISV-208, L-653328, LM-2616, SB-226552, SR-58894A, SR-59230A, TZC-5665, UK-1745, YM-430, and the like. One skilled in the art will appreciate that the β-adrenergic antagonists can be administered in the form of pharmaceutically acceptable salts and/or stereoisomers. Suitable β-adrenergic antagonists are described more fully in the literature, such as in Goodman and Gilman, The Pharmacological Basis of Therapeutics (9th Edition), McGraw-Hill, 1995; and the Merck Index on CD-ROM, ₁₃^{th} Edition; and on STN Express, file phar and file registry.

In some embodiments, the solid dosage formulations further comprise atenolol, bisoprolol, carvedilol, metoprolol, nebivolol, propranolol or timolol. In one embodiment, the formulations further comprise atenolol in an amount of about 50 milligrams to about 200 milligrams. In one embodiment, the formulations further comprise bisoprolol (e.g., bisoprolol fumarate) in an amount of about 2.5 milligrams to about 30 milligrams. In one embodiment, the formulations further comprise carvedilol in an amount of about 3.125 milligrams to about 200 milligrams. In one embodiment, the formulations further comprise metoprolol (e.g., metoprolol tartarate, metoprolol succinate) in an amount of about 20 milligrams to about 300 milligrams. In one embodiment, the formulations further comprise nebivolol (e.g., nebivolol hydrochloride) in an amount of about 2.5 milligrams to about 20 milligrams. In one embodiment, the formulations further comprise propranolol (e.g., propranolol hydrochloride) in an amount of about 40 milligrams to about 240 milligrams. In one embodiment, the formulations further comprise timolol (e.g., timolol maleate) in an amount of about 10 milligrams to about 30 milligrams. In some formulation the β-adrenergic antagonist is preferably metoprolol.

In one embodiment, the solid dosage formulations further comprise an angiotensin II antagonist. Suitable angiotensin II antagonists include, but are not limited to, angiotensin, abitesartan, candesartan, candesartan cilexetil, elisartan, embusartan, enoltasosartan, eprosartan, fonsartan, forasartan, glycyllosartan, irbesartan, losartan, olmesartan, milfasartan, medoxomil, ripisartan, pomisartan, pratosartan, saprisartan, saralasin, sarmesin, tasosartan, telmisartan, valsartan, zolasartan, 3-(2′(tetrazole-5-yl)-1,1′-biphen-4-yl)methyl-5,7-dimethyl-2-ethyl-3H-imidazo[4,5-b]pyridine, antibodies to angiotensin II, A-81282, A-81988, BAY 106734, BIBR-363, BIBS-39, BIBS-222, BMS-180560, BMS-184698, BMS-346567, CGP-38560A, CGP-42112A, CGP-48369, CGP-49870, CGP-63170, CI-996, CP-148130, CL-329167, CV-11194, CV-11974, DA-2079, DE-3489, DMP-811, DuP-167, DuP-532, DuP-753, E-1477, E-4177, E-4188, EMD-66397, EMD-666R4, EMD-73495, EMD-66684, EXP-063, EXP-929, EXP-3134, EXP-3174, EXP-6155, EXP-6803, EXP-7711, EXP-9270, EXP-9954, FK-739, FRI 153332, GA-0050, GA-0056, HN-65021, HOE-720, HR-720, ICI-D6888, ICI-D7155, ICI-D8731, KRI-1177, KT3-671, KT-3579, KW-3433, L-158809, L-158978, L-159282 (MK-996), L-159689, L-159874, L-161177, L-162154, L-162234, L-162441, L-163007, L-163017, LF-70156, LRB-057, LRB-081, LRB-087, LY-235656, LY-266099, LY-285434, LY-301875, LY-302289, LY-315995, ME-3221, MK-954, PD-123177, PD-123319, PD-126055, PD-150304, RG-13647, RWJ-38970, RWJ-46458, S-8307, S-8308, SC-51757, SC-54629, SC-52458, SC-52459, SK 1080, SL-910102, SR-47436, TAK-536, UP-2696, U-96849, U-97018, UK-77778, UP-275-22, WAY-126227, WK-1260, WK-1360, WK-1492, WY 126227, YH-1498, YM-358, YM-31472, X-6803, XH-148, XR-510, ZD-6888, ZD-7155, ZD-8731, ZD 8131, the compounds of ACS registry numbers 133240-46-7, 135070-05-2, 139958-16-0, 145160-84-5, 147403-03-0, 153806-29-2, 439904-54-8P, 439904-55-9P, 439904-56-0P, 439904-57-1P, 439904-58-2P, 155918-60-8P, 155918-61-9P, 272438-16-1P, 272446-75-0P, 223926-77-0P, 169281-89-4, 165113-17-7P, 165113-18-8P, 165113-19-9P, 165113-20-2P, 165113-13-3P, 165113-14-4P, 165113-15-5P, 165113-16-6P, 165113-21-3P, 165113-22-4P, 165113-23-5P, 165113-24-6P, 165113-25-7P, 165113-26-8P, 165113-27-9P, 165113-28-0P, 165113-29-1P, 165113-30-4P, 165113-31-5P, 165113-32-6P, 165113-33-7P, 165113-34-8P, 165113-35-9P, 165113-36-0P, 165113-37-1P, 165113-38-2P, 165113-39-3P, 165113-40-6P, 165113-41-7P, 165113-42-8P, 165113-43-9P, 165113-44-0P, 165113-45-1P, 165113-46-2P, 165113-47-3P, 165113-48-4P, 165113-49-5P, 165113-50-8P, 165113-51-9P, 165113-52-0P, 165113-53-1P, 165113-54-2P, 165113-55-3P, 165113-56-4P, 165113-57-5P, 165113-58-6P, 165113-59-7P, 165113-60-0P, 165113-61-1P, 165113-62-2P, 165113-63-3P, 165113-64-4P, 165113-65-5P, 165113-66-6P, 165113-67-7P, 165113-68-8P, 165113-69-9P, 165113-70-2P, 165113-71-3P, 165113-72-4P, 165113-73-5P, 165113-74-6P, 114798-27-5, 114798-28-6, 114798-29-7, 124749-82-2, 114798-28-6, 124749-84-4, 124750-88-5, 124750-91-0, 124750-93-2, 161946-65-2P, 161947-47-3P, 161947-48-4P, 161947-51-9P, 161947-52-0P, 161947-55-3P, 161947-56-4P, 161947-60-0P, 161947-61-1P, 161947-68-8P, 161947-69-9P, 161947-70-2P, 161947-71-3P, 161947-72-4P, 161947-74-6P, 161947-75-7P, 161947-81-5P, 161947-82-6P, 161947-83-7P, 161947-84-8P, 161947-85-9P, 161947-86-0P, 161947-87-1P, 161947-88-2P, 161947-89-3P, 161947-90-6P, 161947-91-7P, 161947-92-8P, 161947-93-9P, 161947-94-0P, 161947-95-1P, 161947-96-2P, 161947-97-3P, 161947-98-4P, 161947-99-5P, 161948-00-1P, 161948-01-2P, 161948-02-3P, 168686-32-6P, 167301-42-0P, 166813-82-7P, 166961-56-4P, 166961-58-6P, 158872-96-9P, 158872-97-0P, 158807-14-8P, 158807-15-9P, 158807-16-0P, 158807-17-1P, 158807-18-2P, 158807-19-3P, 158807-20-6P, 155884-08-5P, 154749-99-2, 167371-59-7P, 244126-99-6P, 177848-35-0P, 141309-82-2P, and the like. Suitable angiotensin II antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments, the solid dosage formulations further comprise candesartan, eprosartan, irbesartan, losartan, omlesartan, telmisartan or valsartan. In other embodiments, the formulations further comprise candesartan (e.g., candesartan cilexetil) in an amount of about 15 milligrams to about 100 milligrams. In other embodiments, the formulations further comprise eprosartan (e.g., eprosartan mesylate) in an amount of about 400 milligrams to about 1600 milligrams. In other embodiments, the formulations further comprise irbesartan in an amount of about 75 milligrams to about 1200 milligrams. In other embodiments, the formulations further comprise losartan (e.g., losartan potassium) in an amount of about 25 milligrams to about 100 milligrams. In other embodiments, the formulations further comprise omlesartan (e.g., omlesartan medoxomil) in an amount of about 5 milligrams to about 40 milligrams. In other embodiments, the formulations further comprise telmisartan in an amount of about 20 milligrams to about 80 milligrams. In other embodiments, the formulations further comprise valsartan in an amount of about 80 milligrams to about 320 milligrams.

In one embodiment, the solid dosage formulations further comprise an aldosterone antagonist. Suitable aldosterone antagonists include, but are not limited to, canrenone, potassium canrenoate, drospirenone, spironolactone, eplerenone (INSPRA®), epoxymexrenone, fadrozole, pregn-4-ene-7,21-dicarboxylic acid, 9,11-epoxy-17-hydroxy-3-oxo, γ-lactone, methyl ester, (7α,11α,17β)-; pregn-4-ene-7,21-dicarboxylic acid, 9,11-epoxy-17-hydroxy-3-oxo-dimethyl ester, (7α,11α,17β)-; 3′H-cyclopropa(6,7) pregna-4,6-diene-21-carboxylic acid, 9,11-epoxy-6,7-dihydro-17-hydroxy-3-oxo-, γ-lactone, (6β,7β,11α,17β)-; pregn-4-ene-7,21-dicarboxylic acid, 9,11-epoxy-17-hydroxy-3-oxo-, 7-(1-methylethyl) ester, monopotassium salt, (7α,11α,17β)-; pregn-4-ene-7,21-dicarboxylic acid, 9,11,-epoxy-17-hydroxy-3-oxo-, 7-methyl ester, monopotassium salt, (7α,11α,17β)-; 3′H-cyclopropa(6,7) pregna-1,4,6-triene-21-carboxylic acid, 9,11-epoxy-6,7-dihydro-17-hydroxy-3-oxo-, γ-lactone, (6β,7β,11α)-; 3′H-cyclopropa(6,7)pregna-4,6-diene-21-carboxylic acid, 9,11-epoxy-6,7-dihydro-17-hydroxy-3-oxo-, methyl ester, (6β,7β,11α,17β)-; 3′H-cyclopropa (6,7)pregna-4,6-diene-21-carboxylic acid, 9,11-epoxy-6,7-dihydro-17-hydroxy-3-oxo-, monopotassium salt, (6β,7β,11α,17β)-; 3′H-cyclopropa(6,7)pregna-1,4,6-triene-21-carboxylic acid, 9,11-epoxy-6,7-dihydro-17-hydroxy-3-oxo-, γ-lactone, (6β,7β,11α,17β)-; pregn-4-ene-7,21-dicarboxylic acid, 9,11-epoxy-17-hydroxy-3-oxo-, γ-lactone, ethyl ester, (7α,11α,17β)-; pregn-4-ene-7,21-dicarboxylic acid, 9,11-epoxy-17-hydroxy-3-oxo-, γ-lactone, 1-methylethyl ester, (7α,11α,17β)-; RU-28318, and the like. One skilled in the art will appreciate that the aldosterone antagonists can be administered in the form of their pharmaceutically acceptable salts and/or stereoisomers. Suitable aldosterone antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments, the solid dosage formulations further comprise eplerenone and/or spironolactone. In other embodiments, the formulations further comprise eplerenone in an amount of about 25 milligrams to about 300 milligrams. In other embodiments, the formulations further comprise spironolactone in an amount of about 25 milligrams to about 150 milligrams.

In other embodiments, the solid dosage formulations further comprise digials. Exemplary digitals include digoxin and digoxitin. In some embodiments, the formulations further comprise digoxin is an amount to achieve a steady state blood serum concentration of at least about 0.7 nanograms per ml to about 2.0 nanograms per ml.

In some embodiments, the solid dosage formulations further comprise a diuretic. Suitable diuretics include but are not limited to, thiazides (such as, for example, althiazide, bendroflumethiazide, benzclortriazide, benzhydrochlorothiazide, benzthiazide, buthiazide, chlorothiazide, cyclopenethiazide, cyclothiazide, epithiazide, ethiazide, hydrobenzthiazide, hydrochlorothiazide, hydroflumethiazide, methylclothiazide, methylcyclothiazide, penflutazide, polythiazide, teclothiazide, trichlormethiazide, triflumethazide, and the like); alilusem, ambuside, amiloride, aminometradine, azosemide, bemetizide, bumetanide, butazolamide, butizide, canrenone, carperitide, chloraminophenamide, chlorazanil, chlormerodrin, chlorthalidone, cicletanide, clofenamide, clopamide, clorexolone, conivaptan, daglutril, dichlorophenamide, disulfamide, ethacrynic acid, ethoxzolamide, etozolon, fenoldopam, fenquizone, furosemide, indapamide, mebutizide, mefruside, meralluride, mercaptomerin sodium, mercumallylic acid, mersalyl, methazolamide, meticane, metolazone, mozavaptan, muzolimine, N-(5-1,3,4-thiadiazol-2-yl)acetamide, nesiritide, pamabrom, paraflutizide, piretanide, protheobromine, quinethazone, scoparius, spironolactone, theobromine, ticrynafen, torsemide, torvaptan, triamterene, tripamide, ularitide, xipamide or potassium, AT 189000, AY 31906, BG 9928, BG 9791, C 2921, DTI 0017, JDL 961, KW 3902, MCC 134, SLV 306, SR 121463, WAY 140288, ZP 120, and the like. One skilled in the art will appreciate that the diuretics can be administered in the form of their pharmaceutically acceptable salts and/or stereoisomers. Suitable diuretics are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments, the solid dosage formulations further comprise amiloride, furosemide, chlorthalidone, chlorothiazide, hydrochlorothiazide or triamterene. In one embodiment, the formulations further comprises amiloride (e.g., amiloride hydrochloride) in an amount of about 5 milligrams to about 15 milligrams. In one embodiment, the formulations further comprises furosemide in an amount of about 10 milligrams to about 600 milligrams. In one embodiment, the formulations further comprises chlorthalidone in an amount of about 15 milligrams to about 150 milligrams. In one embodiment, the formulations further comprises chlorothiazide in an amount of about 500 milligrams to about 2 grams. In one embodiment, the formulations further comprises triamterene in an amount of about 35 milligrams to about 225 milligrams.

In some embodiments the immediate release, sustained release, delayed release, variable release and/or pulsed release solid dosage formulations further comprise lisinopril, enalapril or captopril. In another embodiment the immediate release, sustained release, delayed release, variable release and/or pulsed release formulations further comprise metoprolol.

The solid dosage formulations described herein may be used in methods for (a) reducing mortality associated with heart failure; (b) improving oxygen consumption; (c) treating heart failure; (d) treating hypertension; (e) improving the quality of life in a heart failure patient; (f) inhibiting left ventricular remodeling; (g) reducing hospitalizations related to heart failure; (h) improving exercise tolerance; (j) increasing left ventricular ejection fraction; (k) decreasing levels of B-type natriuretic protein; (l) treating renovascular diseases; (m) treating end-stage renal diseases; (n) reducing cardiomegaly; (o) treating diseases resulting from oxidative stress; (p) treating endothelial dysfunctions; (q) treating diseases caused by endothelial dysfunctions; (r) treating cardiovascular diseases; (s) treating respiratory disorders; (t) treating blood disorders; (u) treating the symptoms and/or complications associated with blood disorders; (v) treating preeclampsia; by administering to the patient in need thereof an effective amount of the formulations. In one embodiment the solid dosage formulations comprising hydralazine are used for treating hypertension and/or treating preeclampsia.

While individual needs may vary, determination of optimal ranges for effective amounts of the formulations is within the skill of the art and may be determined by standard clinical techniques, including reference to Goodman and Gilman, supra; The Physician's Desk Reference, Medical Economics Company, Inc., Oradell, N.J., 1995; Drug Facts and Comparisons, Inc., St. Louis, Mo., 1993; and the effective amounts described herein. Generally, the dosage required to provide an effective amount of the formulations, which may be adjusted by one of ordinary skill in the art, may vary depending on the age, health, physical condition, sex, diet, weight, extent of the dysfunction of the recipient, frequency of treatment and the nature and scope of the dysfunction or disease, medical condition of the patient, the route of administration, pharmacological considerations such as, the activity, efficacy, pharmacokinetic and toxicology profiles of the particular compound used, whether a drug delivery system is used, and whether the compound is administered as part of a drug combination.

In another embodiment, the invention provides pharmaceutical kits comprising the immediate release, sustained release, delayed release, variable release and/or pulsed release solid dosage formulations. The pharmaceutical kits may also comprise at least one of an angiotensin converting enzyme inhibitor, a β-adrenergic antagonist, an angiotensin II antagonist, an aldosterone antagonist, a digitalis, a diuretic compound, or a combination of two or more thereof. The formulations and the at least one of an angiotensin converting enzyme inhibitor, a β-adrenergic antagonist, an angiotensin II antagonist, an aldosterone antagonist, a digitalis, a diuretic compound, can be separate components in the kit or can be in the form of a composition in one or more pharmaceutically acceptable carriers.

### EXAMPLE

The following example is for purposes of illustration only and is not intended to limit the spirit or scope of the appended claims.

### Example 1

### Immediate Release Formulation Comprising Hydralazine Hydrochloride and Isosorbide Dinitrate

An immediate release formulation comprising hydralazine hydrochloride and isosorbide dinitrate was prepared by pre-blending, compaction, blending, compression and coating described below:

I. Pre-Blend

| | | |
|---|---|---|
| Hydralazine hydrochloride, USP | 101.25 | kg |
| Diluted isosorbide dinitrate | 360.00 | kg |
| (ISDN/MCC) | | |
| Microcrystalline cellulose | 59.55 | kg |
| Sodium starch glycolate, NF | 5.40 | kg |
| Magnesium stearate, NF | 1.35 | kg |
| Milled Colloidal silicon dioxide, | 5.70 | kg |
| NF (2.7 kg)/Microcrystalline | | |
| cellulose, NF (3.0 kg) | | |

II Compaction

The blended material was compacted and milled using an integrated roller compactor/mill.

III. Final Blend

The milled granulation was returned to the blender. Separately 2.70 kg of colloidal silicon dioxide and 3.0 kg of the above compacted/milled granulation were added to the granules already in the blender. Thereafter 4.05 kg of magnesium stearate was added to the blender and the resulting mixture was blended.

IV. Compression

The granules from the final blend were compressed at a target core weight of 200 mg on a power-assisted rotary tablet press. The tablets were debossed N on one side and 20 over the score on the other side.

V. Coating

The resulting tablets were coated with a coating solution of 16.20 kg of OPADRY® Orange, YS-1-6227 and 92.10 kg of purified water, USP using a pan coater equipped with a perforated coating system.

Each of the patents, patent applications, and publications cited herein are incorporated by reference herein in their entirety.

It will be apparent to one skilled in the art that various modifications can be made to the invention without departing from the spirit or scope of the appended claims.

What is claimed is:

1. A solid dosage formulation comprising at least one hydralazine compound or a pharmaceutically acceptable salt thereof and at least one excipient or carrier, wherein the formulation has less than about 0.001% to about 0.1% of a hydrazone compound based on the total weight of the formulation.

2. A solid dosage formulation comprising at least one hydralazine compound or a pharmaceutically acceptable salt thereof, at least one nitric oxide donor compound or a pharmaceutically acceptable salt thereof, and at least one excipient or carrier, wherein the formulation has less than about 0.001% to about 0.1% of a hydrazone compound based on the total weight of the formulation.

3. The formulation of claims 1 or 2, wherein the at least one excipient or carrier is selected from the group consisting of cellulose, microcrystalline cellulose, mannitol and sorbitol.

4. The formulation of claim 1 or 2, wherein the at least one hydralazine compound is hydralazine hydrochloride.

5. The formulation of claim 2, wherein the at least one nitric oxide donor compound is isosorbide dinitrate or isosorbide mononitrate.

6. The formulation of claims 1 or 2, further comprising at least one chelating agent and/or at least one acidic agent.

7. The formulation of claims 1 or 2, wherein the formulation has a water content of about 1% to about 3% based on the total weight of the formulation at the time of manufacture of the formulation.

8. The formulation of claim 1 or 2, wherein the formulation is an immediate release formulation, a sustained release formulation, a delayed release formulation, a variable release formulation, a pulsed release formulation or a combination thereof.

9. The formulation of claim 6, wherein the at least one chelating agent is selected from the group consisting of ethylenediamine tetraacetic acid, ethylene glycol tetraacetic acid, 1,2-bis(o-aminophenoxy)ethane-N,N,N′,N′-tetraacetic acid, nitrilotriacetic acid, ethylenediamine tetraacetic acid disodium salt, or a combination thereof.

10. The formulation of claim 6, wherein the at least one acidic agent is selected from the group consisting of citric acid, fumaric acid, malic acid, ascorbic acid and tartaric acid, lactic acid.

11. A solid dosage formulation comprising hydralazine hydrochloride in an amount of about 30 milligrams to about 400 milligrams and at least one excipient or carrier, wherein the formulation has less than about 0.001% to about 0.1% of a hydrazone compound based on the total weight of the formulation.

12. A solid dosage formulation comprising at least one hydralazine hydrochloride in an amount of about 30 milligrams to about 400 milligrams, isosorbide dinitrate in an amount of about 10 milligrams to about 200 milligrams per day, and at least one excipient or carrier, wherein the formulation has less than about 0.001% to about 0.1% of a hydrazone compound based on the total weight of the formulation.

13. The formulation of claims 11 or 12, wherein the at least one excipient or carrier is selected from the group consisting of cellulose, microcrystalline cellulose, mannitol and sorbitol.

14. The formulation of claims 11 or 12, further comprising at least one chelating agent and/or at least one acidic agent.

15. The formulation of claims 11 or 12, wherein the formulation has a water content of about 1% to about 3% based on the total weight of the formulation at the time of manufacture of the formulation.

16. The formulation of claim 11 or 12, wherein the formulation is an immediate release formulation, a sustained release formulation, a delayed release formulation, a variable release formulation, a pulsed release formulation or a combination thereof.

17. The formulation of claim 14, wherein the at least one chelating agent is selected from the group consisting of ethylenediamine tetraacetic acid, ethylene glycol tetraacetic acid, 1,2-bis(o-aminophenoxy)ethane-N,N,N′,N′-tetraacetic acid, nitrilotriacetic acid, ethylenediamine tetraacetic acid disodium salt, or a combination thereof.

18. The formulation of claim 14, wherein the at least one acidic agent is selected from the group consisting of citric acid, fumaric acid, malic acid, ascorbic acid and tartaric acid, lactic acid.
